(19) United States
(12) Patent Application Publication
(10) Pub. No.: US 2018/0044698 A1
(43) Pub. Date: Feb. 15, 2018

(54) SITE-SPECIFIC INTEGRATING RECOMBINANT AAV VECTORS FOR GENE THERAPY AND IMPROVED PRODUCTION METHODS

(71) Applicant: University of Florida Research Foundation, Incorporated, Gainesville, FL (US)

(72) Inventors: Arun Srivastava, Gainesville, FL (US); Chen Ling, Gainesville, FL (US)

(73) Assignee: University of Florida Research Foundation, Incorporated, Gainesville, FL (US)

(21) Appl. No.: 15/552,163

(22) PCT Filed: Feb. 19, 2016
(86) PCT No.: PCT/US16/18814

§ 371 Date: Aug. 18, 2017

Related U.S. Application Data
(60) Provisional application No. 62/118,125, filed on Feb. 19, 2015.
(60) Provisional application No. 62/118,151, filed on Feb. 19, 2015.
(60) Provisional application No. 62/118,102, filed on Feb. 19, 2015.

(51) Int. Cl. C12N 15/86

(52) U.S. Cl. CPC C12N 15/86; A61K 48/00

(57) ABSTRACT

Provided herein are methods of site-specific integration of a heterologous sequence into a host genome (e.g., by administering a recombinant adeno-associated virus (rAAV) to a host cell in the presence of a Rep protein), and methods for treating diseases and disorders by delivering an rAAV that comprises a nucleic acid vector comprising a Rep protein. Also provided herein are methods and compositions for producing rAAV particles with improved titer and transduction efficiencies.

### RELATED APPLICATIONS

This application claims the benefit under 35 U.S.C. §119(e) of U.S. provisional application No. 62/118,102, filed Feb. 19, 2015, U.S. provisional application No. 62/118,151, filed Feb. 19, 2015, and U.S. provisional application No. 62/118,125, filed Feb. 19, 2015, the contents of each of which are incorporated herein by reference in their entirety.

### GOVERNMENT SUPPORT

This invention was made with government support under HL-097088 and EB-015684 awarded by the National Institutes of Health. The government has certain rights in the invention.

### BACKGROUND

Unlike wild-type adeno-associated virus (AAV), recombinant AAV (rAAV) lacks the ability to integrate into a host genome in a site-specific manner. There remains a need to develop effective compositions and methods to effectively restore site-specific integration with rAAV. Additionally, it is difficult to achieve high yield of rAAV with typical rAAV production procedures. Accordingly, methods are needed for producing rAAV with higher titer and increased transduction efficiency.

### SUMMARY

Provided herein are recombinant AAV (rAAV) particles or preparations, nucleic acid vectors, and methods of use thereof for achieving site-specific integration of a heterologous sequence. Also provided herein are methods and compositions useful in the production of rAAV particles, wherein the methods and compositions provide increased particle titers and/or particles having enhanced transduction efficiencies.

In some aspects, the methods and compositions provided herein are useful in gene therapy. In some aspects, the disclosure provides methods and compositions useful in the treatment of proliferative diseases (e.g., cancer) and/or hematopoietic disorders (e.g., hemoglobinopathies). In other aspects, the disclosure provides methods and compositions useful in the production of rAAV particles (e.g., increased particle titers, increased transduction efficiencies).

In some aspects, the disclosure provides a method of promoting site-specific nucleic acid integration into a host genome, the method comprising: delivering a recombinant adeno-associated virus (rAAV) particle comprising a nucleic acid vector to a host cell in the presence of a Rep protein.

In some embodiments, the nucleic acid vector comprises AAV2 inverted terminal repeats (ITRs) or AAV6 ITRs. In some embodiments, the rAAV particle is a AAV6 particle. In some embodiments, the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination thereof. In some embodiments, the modified capsid protein comprises a non-tyrosine residue and/or a non-threonine residue at one or more of or each of Y705, Y731, and T492 of a wild-type AAV6 capsid protein. In some embodiments, the non-tyrosine residue is phenylalanine and the non-threonine residue is valine.

In some embodiments, the host cell is a human cell. In some embodiments, the host cell is a stem cell. In some embodiments, the host cell is a liver, muscle, brain, eye, pancreas, kidney, or hematopoietic stem cell. In some embodiments, the host cell is ex vivo. In some embodiments, the host cell is in situ in a host. In some embodiments, the host cell is in situ in a host and the rAAV is administered to the host to target one or more host cells.

In some embodiments, the nucleic acid vector encodes the Rep protein. In some embodiments, the Rep protein is delivered to the host cell separately from the nucleic acid vector. In some embodiments, the Rep protein is expressed from a second nucleic acid that is delivered to the host cell. In some embodiments, the second nucleic acid is an mRNA that is transiently transfected into the host cell. In some embodiments, the second nucleic acid is transfected into the host cell in a viral particle.

In some embodiments, the AAV nucleic acid vector encodes a therapeutic protein. In some embodiments, the therapeutic protein is human β-globin.

In some embodiments, the Rep protein is an AAV2 or AAV6 Rep protein.

Some aspects of the disclosure relate to methods for treating proliferative diseases, e.g., by administering a rAAV particle comprising a nucleic acid vector that encodes a Rep protein to a subject having a proliferative disease.

In some aspects, the disclosure provides a method of treating a proliferative disease, the method comprising administering an rAAV particle comprising a nucleic acid vector that encodes a Rep protein to a subject having the proliferative disease. In some embodiments, the rAAV particle is a recombinant AAV3, AAV5, or AAV6 particle. In some embodiments, the Rep protein is an AAV3, AAV5, or AAV6 Rep protein. In some embodiments, the nucleic acid vector comprises AAV3 inverted terminal repeats (ITRs), AAV5 ITRs, or AAV6 ITRs.

In some embodiments, the proliferative disease is cancer. In some embodiments, the cancer is liver cancer.

In some embodiments, the nucleic acid vector comprises a human alpha-fetoprotein (AFP) promoter. In some embodiments, the nucleic acid vector further encodes a therapeutic protein or nucleic acid. In some embodiments, the therapeutic protein or nucleic acid is selected from a caspase, Bcl2, BAX, p53, retinoblastoma (RB), thymidine kinase (TK), pyruvate dehydrogenase (PDH) E1α, β-catenin/Yes-associated protein 1 (YAP1)-siRNA, survivin siRNA, Parvovirus B19 non-structural protein 1 (NS1) and trichosanthin (TCS).

Some aspects of the disclosure relate to methods of producing rAAV particles with higher particle titer and increased transduction efficiency. In some embodiments, the rAAV particles are produced by packaging a nucleic acid vector comprising inverted terminal repeat (ITR) sequences of a selected serotype in the presence of a Rep protein of the same serotype.

In some aspects, the disclosure relates to methods of preparing rAAV composition by packaging a recombinant AAV nucleic acid vector comprising ITRs of a first serotype in the presence of a) a Rep protein of the same serotype and b) AAV capsid proteins. As described herein, it has been found that use of ITRs and Rep proteins from AAV3 to package rAAV particles resulted in both higher titer of the particles produced and a higher transduction efficiency of the produced particles.

In some aspects, the disclosure relates to a method of producing an rAAV composition, the method comprising packaging a recombinant AAV nucleic acid vector comprising ITRs of a first serotype in the presence of a) a Rep protein of the same serotype and b) AAV capsid proteins, wherein the first serotype is not AAV2 or AAV8. In some embodiments, the AAV capsid proteins are of the same serotype as the ITRs and Rep protein. In some embodiments, the first serotype is AAV3, AAV5 or AAV6. In some embodiments, the first serotype is AAV1, AAV2, or AAV4.

In some embodiments, the recombinant AAV nucleic acid vector encodes a therapeutic protein. In some embodiments, the therapeutic protein is selected from the group consisting of adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphorins, semaphorin receptors, serotonin transport proteins, serotonin uptake inhibitors, serotonin receptors, serpins, serpin receptors, and tumor suppressors.

In some embodiments, the recombinant AAV nucleic acid vector (e.g., comprising ITRs of a first serotype) encodes a gene of interest (e.g., a therapeutic gene or a gene encoding a therapeutic protein) and a Rep protein (e.g., of the same serotype or of a different serotype as the ITRs). In some embodiments, the recombinant AAV nucleic acid vector comprising ITRs of a first serotype encodes a gene of interest and does not further encode a Rep protein.

In some embodiments, the packaging is performed in a helper cell. In some embodiments, the packaging is performed in vitro.

In some embodiments, the methods of rAAV production provided herein are useful for generating rAAV particles of higher titer and/or enhanced transduction efficiency for use in the treatment of hematopoietic disorders (e.g., hemoglobinopathies). In some embodiments, methods of rAAV production provided herein are useful for generating rAAV particles of higher titer and enhanced transduction efficiency for use in the treatment of proliferative diseases (e.g., cancer).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 illustrates non-limiting embodiments of no or random AAV (e.g., AAV6) integration into a host genome (FIG. 1A) and Rep-mediated site-specific AAV (e.g., AAV6) integration into a host genome (FIG. 1B).

FIG. 2 depicts a series of diagrams showing exemplary gene expression and integration in the presence or absence of a Rep protein. FIG. 2A shows exemplary AAV vector-mediated transient transgene expression in cancer cells, the therapeutic benefit of which is lost due to rapid cellular proliferation and dilution of the vector genomes since the AAV genomes fail to integrate into chromosomal DNA. FIG. 2B shows that exemplary AAV Rep proteins not only induce cytotoxicity, but also mediate stable integration of the AAV genomes into cancer cells, and thus continue to provide therapeutic benefit.

FIG. 3 depicts schematic structures of AAV helper genomes. Arrows indicate the positions of known promoters of AAV2.

FIG. 4 depicts non-limiting experimental data of the effect of ITRs on viral genome replication and encapsidation. HEK293 cells were transfected with the indicated plasmids and pHelper, in the presence of either pAAVr2c3 (FIG. 4A) or pAAVr3c3 (FIG. 4B). HEK293 cells were transfected with ITR2-EGFP-Neo and AAVr2c3, and with ITR3-EGFP-Neo and AAVr3c3 (FIG. 4C). HEK293 cells were alternatively transfected with ITR2-EGFP-Neo and AAVr2c3-S663V+T492V, and with ITR3-EGFP-Neo and AAVr3c3-S663V+T492V (FIG. 4D). Transduction efficiencies of Rep2/ITR2 and Rep3/ITR3 in Huh7 cells were compared (FIG. 4E).

FIG. 5 depicts non-limiting experimental data of transduction efficiency of AAV3 vectors in vitro. WT-AAV3 vectors (FIGS. 5A and 5B) and AAV3-S663V+T492V vectors (FIGS. 5C and 5D) were produced by triple-transfection with either the combination of Rep2/ITR2, or the combination of Rep3/ITR3. Human hepatocellular carcinoma cell lines, Huh7 and LH86, were transduced with the indicated viral vectors, and transgene expression was determined by fluorescence microscopy. Both representative (5A and 5C) and quantitative (5B and 5D) results are shown.

FIG. 6 depicts non-limiting experimental data of transduction efficiency of AAV3 vectors in vivo. EGFP expression in each tumor was determined by Western blotting 48 hours post-vector administration (FIG. 6A). A tumor without vector injection (lane 1) was used as a negative control, and β-actin was used as a loading control. Quantitation of EGFP expression in each tumor is also depicted, normalized with β-actin expression (FIG. 6B).

### DETAILED DESCRIPTION

Aspects of the application relate to methods and compositions useful in gene therapy with recombinant adeno-associated virus (rAAV). In some aspects, the methods and compositions provided herein are useful in promoting site-specific integration of a gene of interest into a host cell genome. In some aspects, the disclosure provides methods and compositions useful in the treatment of proliferative diseases (e.g., cancer) and/or hematopoietic disorders. In other aspects, the disclosure provides methods and compositions useful in the production of rAAV particles with high titers and increased transduction efficiencies.

In some aspects, provided herein are methods, rAAV particles, nucleic acid vectors, and Rep proteins for delivering a heterologous nucleic acid region to a host genome in a site-specific manner.

In some aspects, the disclosure provides a method of promoting site-specific nucleic acid integration into a host genome. In some embodiments, the method comprises delivering a recombinant adeno-associated virus (rAAV) particle as described herein comprising a nucleic acid vector as described herein to a host cell in the presence of a Rep protein.

Any host cell is contemplated for use in a method described herein. In some embodiments, the host cell is a cell in situ in a host, such as a subject as described herein. In some embodiments, the host cell is ex vivo, e.g., in a culture of host cells. In some embodiments, the host cell is a human cell, a non-human primate cell, a dog cell, a cat cell, a mouse cell, a rat cell, a guinea pig cell, or a hamster cell.

In some embodiments, the host cell is a stem cell, such as a hematopoietic stem cell (e.g., a human hematopoietic stem cell). In some embodiments, the host cell is a liver cell, muscle cell, brain cell, eye cell, pancreas cell, or kidney cell.

In some aspects, the disclosure relates to methods of preparing rAAV composition by packaging a recombinant AAV nucleic acid vector comprising ITRs of a first serotype in the presence of a) a Rep protein of the same serotype and b) AAV capsid proteins. As described herein, it has been found that use of ITRs and Rep proteins from AAV3 to package rAAV particles resulted in both higher titer of the particles produced and a higher transduction efficiency of the produced particles.

In some aspects, the disclosure relates to a method of producing an rAAV composition. In some embodiments, the method comprises packaging a nucleic acid vector comprising ITRs of a first serotype in the presence of a) a Rep protein of the same serotype and b) AAV capsid proteins. In some embodiments, the first serotype is not AAV2 or AAV8. In some embodiments, the first serotype is AAV3, AAV5 or AAV6. In some embodiments, the AAV capsid proteins are of the same serotype as the ITRs and Rep protein.

The disclosure also provides compositions comprising one or more of the disclosed nucleic acid vectors, Rep proteins, or rAAV particles. As described herein, such compositions may further comprise a pharmaceutical excipient, buffer, or diluent, and may be formulated for administration to host cell ex vivo or in situ in an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vector genomes(vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In one embodiment, rAAV particles of higher than 10¹³ vgs/mL are be administered. The rAAV particles can be administered as a single dose, or divided into two or more administrations as may be required to achieve therapy of the particular disease or disorder being treated. In some embodiments, 0.0001 mL to 10 mLs are delivered to a subject.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation, Rep proteins, and nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations, Rep proteins, and nucleic acid vectors may thus be delivered along with various other agents as required in the particular instance. Such compositions may be purified from host cells or other biological sources, or alternatively may be chemically synthesized as described herein. As used herein, the term “vector” can refer to a nucleic acid vector (e.g., a plasmid or recombinant viral genome) or a viral vector (e.g., an rAAV particle comprising a recombinant genome).

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV particles or preparations, Rep proteins, and/or nucleic acid vectors in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle or preparation, Rep protein, or nucleic acid vector is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells transduced with rAAV particles or preparations, and/or Rep proteins is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles or preparations, and/or Rep proteins may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the invention.

The amount of rAAV particle or preparation, Rep protein, or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

The composition may include rAAV particles or preparations, Rep proteins, and/or nucleic acid vectors, either alone, or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Recombinant AAV (rAAV) Particles, Preparations, and Nucleic Acid Vectors

Aspects of the disclosure relate to recombinant adeno-associated virus (rAAV) particles or preparations of such particles for delivery of one or more nucleic acid vectors comprising a sequence encoding a Rep protein, and/or a protein or polypeptide of interest, into various tissues, organs, and/or cells. In some embodiments, the rAAV particle is delivered to a host cell in the presence of a Rep protein as described herein.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector, which may comprise at a minimum: (a) one or more heterologous nucleic acid regions comprising a sequence encoding a protein or polypeptide of interest (e.g., a globin gene) or an RNA of interest (e.g., a siRNA or microRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein as described herein. Any nucleic acid vector described herein may be encapsidated by a viral capsid, such as an AAV6 capsid or any other serotype (e.g., a serotype that is of the same serotype as the ITR sequences), which may comprises a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Accordingly, in some embodiments, an rAAV particle or rAAV preparation containing such particles comprises a viral capsid and a nucleic acid vector as described herein, which is encapsidated by the viral capsid. In some embodiments, the nucleic acid vector comprises (1) one or more heterologous nucleic acid regions comprising a sequence encoding a protein or polypeptide of interest (e.g., a globin gene), (2) one or more nucleic acid regions comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more heterologous nucleic acid regions comprising a sequence encoding a protein or polypeptide of interest operably linked to a promoter, wherein the one or more heterologous nucleic acid regions are flanked on each side with an ITR sequence. In some embodiments, the nucleic acid vector further comprises a region encoding a Rep protein as described herein, either contained within the region flanked by ITRs or outside the region. In some embodiments, the nucleic acid vector comprises (1) one or more heterologous nucleic acid regions comprising a sequence encoding a protein or polypeptide of interest, (2) one or more nucleic acid regions comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences of a first serotype. In some embodiments, the nucleic acid vector comprises one or more heterologous nucleic acid regions comprising a sequence encoding a protein or polypeptide of interest operably linked to a promoter, wherein the one or more heterologous nucleic acid regions are flanked on each side with an ITR sequence of a first serotype. In some embodiments, the nucleic acid vector comprises (1) one or more nucleic acid regions comprising a sequence encoding a Rep protein (optionally further comprising a sequence encoding a therapeutic protein or nucleic acid), (2) one or more nucleic acid regions comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more nucleic acid regions comprising a sequence encoding a Rep protein (optionally further comprising a sequence encoding a therapeutic protein or nucleic acid) operably linked to a promoter, wherein the one or more nucleic acid regions are flanked on each side with an ITR sequence. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, a first serotype provided herein is not a AAV2 or AAV8 serotype. In some embodiments, the ITR sequences of the first serotype are derived from AAV3, AAV5 or AAV6. In some embodiments, the ITR sequences are derived from AAV2, AAV3, AAV5 or AAV6. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™ Viral Vectors for Gene TherapyMethods and Protocols. 10.1385/1-59259-304-6:201® Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

Exemplary ITR sequences are provided below.

AAV2:
(SEQ ID NO: 1)
TTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGA
GCGAGCGAGCGCGCAGAGAGGGAGTGGCCAACTCCATCACTAGGGGT
TCCT
AAV3:
(SEQ ID NO: 2)
TTGGCCACTCCCTCTATGCGCACTCGCTCGCTCGGTGGGGCCTGGCG
ACCAAAGGTCGCCAGACGGACGTGCTTTGCACGTCCGGCCCCACCGA
GCGAGCGAGTGCGCATAGAGGGAGTGGCCAACTCCATCACTAGAGGT
ATGGC
AAV5:
(SEQ ID NO: 3)
CTCTCCCCCCTGTCGCGTTCGCTCGCTCGCTGGCTCGTTTGGGGGGG
TGGCAGCTCAAAGAGCTGCCAGACGACGGCCCTCTGGCCGTCGCCCC
CCCAAACGAGCCAGCGAGCGAGCGAACGCGACAGGGGGGAGAGTGCC
ACACTCTCAAGCAAGGGGGTTTTGTA
AAV6:
(SEQ ID NO: 4)
TTGCCCACTCCCTCTATGCGCGCTCGCTCGCTCGGTGGGGCCTGCGG
ACCAAAGGTCCGCAGACGGCAGAGCTCTGCTCTGCCGGCCCCACCGA
GCGAGCGAGCGCGCATAGAGGGAGTGGGCAACTCCATCACTAGGGGT
A

Any Rep protein is contemplated for use in a method described herein. In some embodiments, the Rep protein is an AAV1, AAV2, AAV3, AAV4, AAV5, AVV6, AAV7, AAV8, AAV9, or AAV10 Rep protein or variant thereof. In some embodiments, the Rep protein is an AAV2, AAV3, AAV5, or AAV6 Rep protein or variant thereof. In some embodiments, the Rep protein is an AAV2 or AAV6 Rep protein or variant thereof. In some embodiments, the Rep protein is an AAV6 Rep protein or variant thereof.

Exemplary Rep protein sequences are provided below.

| AAV2 Rep: | |
|---|---|
| (SEQ ID NO: 5) | |
| 1 | TAGFYEIVIK VPSDLDGHLP GISDSFVNWV AEKEWELPPD SDMDLNLIEQ |
| 51 | APLTVAEKLQ RDFLTEWRRV SKAPEALFFV QFEKGESYFH MHVLVETTGV |
| 101 | KSMVLGRFLS QIREKLIQRI YRGIEPTLPN WFAVTKTRNG AGGGNKVVDE |
| 151 | CYIPNYLLPK TQPELQWAWT NMEQYLSACL NLTERKRLVA QHLTHVSQTQ |
| 201 | EQNKENQNPN SDAPVIRSKT SARYMELVGW LVDKGITSEK QWIQEDQASY |
| 251 | ISFNAASNSR SQIKAALDNA GKIMSLTKTA PDYLVGQQPV EDISSNRIYK |
| 301 | ILELNGYDPQ YAASVFLGWA TKKFGKRNTI WLFGPATTGK TNIAEAIAHT |
| 351 | VPFYGCVNWT NENFPFNDCV DKMVIWWEEG KMTAKVVESA KAILGGSKVR |
| 401 | VDQKCKSSAQ IDPTPVIVTS NTNMCAVIDG NSTTFEHQQP LQDRMFKFEL |
| 451 | TRRLDHDFGK VTKQEVKDFF RWAKDHVVEV EHEFYVKKGG AKKRPAPSDA |
| 501 | DISEPKRVRE SVAQPSTSDA EASINYADRY QNKCSRHVGM NLMLFPCRQC |
| 551 | ERMNQNSNIC FTHGQKDCLE CFPVSESQPV SVVKKAYQKL CYIHHIMGKV |
| 601 | PDACTACDLV NVDLDDCIFE Q* |
| AAV3 Rep: | |
| (SEQ ID NO: 6) | |
| 1 | MPGFYEIVLK VPSDLDERLP GISNSFVNWV AEKEWDVPPD SDMDPNLIEQ |
| 51 | APLTVAEKLQ REFLVEWRRV SKAPEALFFV QFEKGETYFH LHVLIETIGV |
| 101 | KSMVVGRYVS QIKEKLVTRI YRGVEPQLPN WFAVTKTRNG AGGGNKVVDD |
| 151 | CYIPNYLLPK TQPELQWAWT NMDQYLSACL NLAERKRLVA QHLTHVSQTQ |
| 201 | EQNKENQNPN SDAPVIRSKT SARYMELVGW LVDRGITSEK QWIQEDQASY |
| 251 | ISFNAASNSR SQIKAALDNA SKIMSLTKTA PDYLVGSNPP EDITKNRIYQ |
| 301 | ILELNGYDPQ YAASVFLGWA QKKFGKRNTI WLFGPATTGK TNIAEAIAHA |
| 351 | VPFYGCVNWT NENFPFNDCV DKMVIWWEEG KMTAKVVESA KAILGGSKVR |
| 401 | VDQKCKSSAQ IEPTPVIVTS NTNMCAVIDG NSTTFEHQQP LQDRMFEFEL |
| 451 | TRRLDHDFGK VTKQEVKDFF RWASDHVTDV AHEFYVRKGG AKKRPASNDA |
| 501 | DVSEPKRECT SLAQPTTSDA EAPADYADRY QNKCSRHVGM NLMLFPCKTC |
| 551 | ERMNQISNVC FTHGQRDCGE CFPGMSESQP VSVVKKKTYQ KLCPIHHILG |
| 601 | RAPEIACSAC DLANVDLDDC VSEQ* |
| AAV5 Rep: | |
| (SEQ ID NO: 7) | |
| 1 | MATFYEVIVR VPFDVEEHLP GISDSFVDWV TGQIWELPPE SDLNLTLVEQ |
| 51 | PQLTVADRIR RVFLYEWNKF SKQESKFFVQ FEKGSEYFHL HTLVETSGIS |
| 101 | SMVLGRYVSQ IRAQLVKVVF QGIEPQINDW VAITKVKKGG ANKVVDSGYI |
| 151 | PAYLLPKVQP ELQWAWTNLD EYKLAALNLE ERKRLVAQFL AESSQRSQEA |
| 201 | ASQREFSADP VIKSKTSQKY MALVNWLVEH GITSEKQWIQ ENQESYLSFN |
| 251 | STGNSRSQIK AALDNATKIM SLTKSAVDYL VGSSVPEDIS KNRIWQIFEM |
| 301 | NGYDPAYAGS ILYGWCQRSF NKRNTVWLYG PATTGKTNIA EAIAHTVPFY |
| 351 | GCVNWTNENF PFNDCVDKML IWWEEGKMTN KVVESAKAIL GGSKVRVDQK |
| 401 | CKSSVQIDST PVIVTSNTNM CVVVDGNSTT FEHQQPLEDR MFKFELTKRL |
| 451 | PPDFGKITKQ EVKDFFAWAK VNQVPVTHEF KVPRELAGTK GAEKSLKRPL |
| 501 | GDVTNTSYKS LEKRARLSFV PETPRSSDVT VDPAPLRPLN WNSRYDCKCD |
| 551 | YHAQFDNISN KCDECEYLNR GKNGCICHNV THCQICHGIP PWEKENLSDF |
| 601 | GDFDDANKEQ* |
| AAV6 Rep | |
| (SEQ ID NO: 8) | |
| 1 | MPGFYEIVIK VPSDLDEHLP GISDSFVNWV AEKEWELPPD SDMDLNLIEQ |
| 51 | APLTVAEKLQ RDFLVQWRRV SKAPEALFFV QFEKGESYFH LHILVETTGV |
| 101 | KSMVLGRFLS QIRDKLVQTI YRGIEPTLPN WFAVTKTRNG AGGGNKVVDE |
| 151 | CYIPNYLLPK TQPELQWAWT NMEEYISACL NLAERKRLVA HDLTHVSQTQ |
| 201 | EQNKENLNPN SDAPVIRSKT SARYMELVGW LVDRGITSEK QWIQEDQASY |
| 251 | ISFNAASNSR SQIKAALDNA GKIMALTKSA PDYLVGPAPP ADIKTNRIYR |
| 301 | ILELNGYDPA YAGSVFLGWA QKRFGKRNTI WLFGPATTGK TNIAEAIAHA |
| 351 | VPFYGCVNWT NENFPFNDCV DKMVIWWEEG KMTAKVVESA KAILGGSKVR |
| 401 | VDQKCKSSAQ IDPTPVIVTS NTNMCAVIDG NSTTFEHQQP LQDRMFKFEL |
| 451 | TRRLEHDFGK VTKQEVKEFF RWAQDHVTEV AHEFYVRKGG ANKRPAPDDA |
| 501 | DKSEPKRACP SVADPSTSDA EGAPVDFADR YQNKCSRHAG MLQMLFPCKT |
| 551 | CERMNQNFNI CFTHGTRDCS ECFPGVSESQ PVVRKRTYRK LCAIHHLLGR |
| 601 | APEIACSACD LVNVDLDDCV SEQ* |

The Rep protein may be delivered to the host cell in any suitable manner. In some embodiments, the Rep protein is delivered at the same time as the rAAV particle, e.g., where the nucleic acid vector encodes the Rep protein or the Rep protein is delivered as a protein, as an mRNA encoding the Rep protein, or as a second nucleic acid vector encoding the Rep protein (e.g., within a second rAAV particle). Methods for producing rAAV particles and nucleic acid vectors are described herein. In some embodiments, if the Rep protein is delivered as an mRNA encoding the Rep protein, the mRNA is delivered to the host cell, e.g., using a transient transfection method. Exemplary transient transfection methods and reagents are known in the art and include, e.g., use of Lipofectamine® reagents, electroporation, liposomes, lipids or lipid complexes, microspheres, microparticles, nanospheres, and nanoparticles.

The rAAV particle, nucleic acid vector, and/or Rep protein (in any form contemplated herein) may be delivered in the form of a composition, such as a composition comprising the active ingredient, such as the rAAV particle, nucleic acid vector, and/or Rep protein (in any form contemplated herein), and a therapeutically or pharmaceutically acceptable carrier. The rAAV particles, Rep proteins, or nucleic acid vectors may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable carrier. The rAAV particles or preparations may be prepared in a variety of compositions, and may also be formulated in appropriate pharmaceutical vehicles for administration to human or animal subjects.

In some embodiments, the nucleic acid vector comprises one or more regions comprising a sequence that facilitates expression of the nucleic acid (e.g., the heterologous nucleic acid or the nucleic acid region encoding the Rep protein), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include hematopoietic stem cell-specific promoters.

Synthetic promoters are also contemplated herein. A synthetic promoter may comprise, for example, regions of known promoters, regulatory elements, transcription factor binding sites, enhancer elements, repressor elements, and the like.

In some embodiments, the promoter is a human alpha-fetoprotein (AFP) promoter. An exemplary human AFP promoter sequence is provided below. In some embodiments, the human AFP promoter is a SV40-AFP promoter. An exemplary SV40-AFP promoter sequence is provided below.

hAFP promoter sequence
(SEQ ID NO: 9)
AGTTTGAGGAGAATATTTGTTATATTTGCAAAATAAAATAAGTTTGCA
AGTTTTTTTTTTCTGCCCCAAAGAGCTCTGTGTCCTTGAACATAAAAT
ACAAATAACCGCTATGCTGTTAATTATTGGCAAATGTCCCATTTTCAA
CCTAAGGAAATACCATAAAGTAACAGATATACCAACAAAAGGTTACTA
GTTAACAGGCATTGCCTGAAAAGAGTATAAAAGAATTTCAGCATGATT
TTCCATATTGTGCTTCCACCACTGCCAATAACACC
SV40-hAFP promoter sequence
(SEQ ID NO: 10)
GGCCTGAAATAACCTCTGAAAGAGGAACTTGGTTAGGTACCTTCTGAG
GCTGAAAGAACCAGCTGTGGAATGTGTGTCAGTTAGGGTGTGGAAAGT
CCCCAGGCTCCCCAGCAGGCAGAAGTATGCAAAGCATGCATCTCAATT
AGTCAGCAACCAGGTGTGGAAAGTCCCCAGGCTCCCCAGCAGGCAGAA
GTATGCAAAGCATGCATCTCAATTAGTCAGCAACCATAGTCCCAGTTT
GAGGAGAATATTTGTTATATTTGCAAAATAAAATAAGTTTGCAAGTTT
TTTTTTTCTGCCCCAAAGAGCTCTGTGTCCTTGAACATAAAATACAAA
TAACCGCTATGCTGTTAATTATTGGCAAATGTCCCATTTTCAACCTAA
GGAAATACCATAAAGTAACAGATATACCAACAAAAGGTTACTAGTTAA
CAGGCATTGCCTGAAAAGAGTATAAAAGAATTTCAGCATGATTTTCCA
TATTGTGCTTCCACCACTGCCAATAACACC

In some embodiments, a nucleic acid vector described herein may also contain marker or reporter genes, e.g., LacZ or a fluorescent protein.

In some embodiments, the nucleic acid vector comprises one or more heterologous nucleic acid regions comprising a sequence encoding a protein or polypeptide of interest, such as a therapeutic protein provided in Table 1 or described herein.

In some embodiments, the nucleic acid vector further comprises a sequence encoding a therapeutic protein or nucleic acid (e.g., a siRNA or microRNA). In some embodiments, the therapeutic protein or nucleic acid is selected from a caspase, Bcl2, BAX, p53, retinoblastoma (RB), thymidine kinase (TK), pyruvate dehydrogenase (PDH) E1α, β-catenin/Yes-associated protein 1 (YAP1)-siRNA, survivin siRNA, Parvovirus B19 non-structural protein 1 (NS1) and trichosanthin (TCS) (see, e.g., Mol. Genet. Metabol., 98: 289-299, 2009 (PDH-E1α); Human Gene Therapy, 25: 1023-1034, 2014 (TCS); Gastroenterology, 147: 690-701, 2014 (β-catenin/Yes-associated protein 1 (YAP1)-siRNA); Human gene Therapy, 26: 94-103, 2015 (TK).).

In some embodiments, the therapeutic protein is a globin protein. Exemplary globin proteins include, but are not limited to, a β-globin (e.g., human β-globin), an anti-sickling β-globin gene (e.g., a human anti-sickling β-globin gene), and a γ-globin gene (e.g., a human γ-globin gene). Exemplary protein sequences for each globin gene mentioned above are provided below.

Human β-globin protein:
(SEQ ID NO: 11)
MVHLTPEEKSAVTALWGKVNVDEVGGEALGRLLVVYPWTQRFFESFGD
LSTPDAVMGNPKVKAHGKKVLGAFSDGLAHLDNLKGTFATLSELHCDK
LHVDPENFRLLGNVLVCVLAHHFGKEFTPPVQAAYQKVVAGVANALAH
KYH
Human γ-globin protein:
(SEQ ID NO: 12)
MGHFTEEDKATITSLWGKVNVEDAGGETLGRLLVVYPWTQRFFDSFGN
LSSASAIMGNPKVKAHGKKVLTSLGDAIKHLDDLKGTFAQLSELHCDK
LHVDPENFKLLGNVLVTVLAIHFGKEFTPEVQASWQKMVTGVASALSS
RYH
Human anti-sickling β-globin gene protein sequence:
(SEQ ID NO: 13)
MVHLTPEEKSAVTALWGKVNVDEVGGEALGRLLVVYPWTQRFFESFGD
LSTPDAVMGNPKVKAHGKKVLGAFSDGLAHLDNLKGTFAQLSELHCDK
LHVDPENFRLLGNVLVCVLAHHFGKEFTPPVQAAYQKVVAGVANALAH
KYH

The protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications provided in Table 1.

TABLE 1
Non-limiting examples of proteins or polypeptides
of interest and associated diseases

| | | Non-limiting NCBI |
|---|---|---|
| Protein or | Non-limiting | Protein IDs or |
| Polypeptide | Exemplary diseases | Patent SEQ ID NOs |
| acid alpha- | Pompe | NP_000143.2, |
| glucosidase (GAA) | | NP_001073271.1, |
| | | NP_001073272.1 |
| Methyl CpG binding | Rett syndrome | NP_001104262.1, |
| protein 2 (MECP2) | | NP_004983.1 |
| Aromatic L-amino | Parkinson's | NP_000781.1, |
| acid decarboxylase | disease | NP_001076440.1, |
| (AADC) | | NP_001229815.1, |
| | | NP_001229816.1, |
| | | NP_001229817.1, |
| | | NP_001229818.1, |
| | | NP_001229819.1 |
| Glial cell-derived | Parkinson's | NP_000505.1, |
| neurotrophic factor | disease | NP_001177397.1, |
| (GDNF) | | NP_001177398.1, |
| | | NP_001265027.1, |
| | | NP_954701.1 |
| Cystic fibrosis | Cystic fibrosis | NP_000483.3 |
| transmembrane | | |
| conductance regulator | | |
| (CFTR) | | |
| Tumor necrosis factor | Arthritis, | SEQ ID NO. 1 of |
| receptor fused | Rheumatoid | WO2013025079 |
| to an antibody Fc | arthritis | |
| (TNFR:Fc) | | |
| HIV-1 gag-proΔrt | HIV infection | SEQ ID NOs. 1-5 of |
| (tgAAC09) | | WO2006073496 |
| Sarcoglycan alpha, | Muscular | SGCA |
| beta, gamma, delta, | dystrophy | NP_000014.1, |
| epsilon, or zeta | | NP_001129169.1 |
| (SGCA, SGCB, | | SGCB |
| SGCG, SGCD, | | NP_000223.1 |
| SGCE, or SGCZ) | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Alpha-1-antitrypsin | Hereditary | NP_000286.3, |
| (AAT) | emphysema or | NP_001002235.1, |
| | Alpha-1- | NP_001002236.1, |
| | antitrypsin | NP_001121172.1, |
| | deficiency | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Glutamate | Parkinson's | NP_000808.2, |
| decarboxylase | disease | NP_038473.2 |
| 1(GAD1) | | |
| Glutamate | Parkinson's | NP_000809.1, |
| decarboxylase | disease | NP_001127838.1 |
| 2 (GAD2) | | |
| Aspartoacylase | Canavan's | NP_000040.1, |
| (ASPA) | disease | NP_001121557.1 |
| Nerve growth | Alzheimer's | NP_002497.2 |
| factor (NGF) | disease | |
| Granulocyte-macrophage | Prostate | NP_000749.2 |
| colonystimulating | cancer | |
| factory (GM-CSF) | | |
| Cluster of | Malignant | NP_001193853.1, |
| Differentiation | melanoma | NP_001193854.1, |
| 86 (CD86 or | | NP_008820.3, |
| B7-2) | | NP_787058.4, |
| | | NP_795711.1 |
| Interleukin 12 | Malignant | NP_000873.2, |
| (IL-12) | melanoma | NP_002178.2 |
| neuropeptide | Parkinson's | NP_000896.1 |
| Y (NPY) | disease, | |
| | epilepsy | |
| ATPase, Ca++ | Chronic heart | NP_001672.1, |
| transporting, cardiac | failure | NP_733765.1 |
| muscle, slow twitch | | |
| 2 (SERCA2) | | |
| Dystrophin or | Muscular | NP_000100.2, |
| Minidystrophin | dystrophy | NP_003997.1, |
| | | NP_004000.1, |
| | | NP_004001.1, |
| | | NP_004002.2, |
| | | NP_004003.1, |
| | | NP_004004.1, |
| | | NP_004005.1, |
| | | NP_004006.1, |
| | | NP_004007.1, |
| | | NP_004008.1, |
| | | NP_004009.1, |
| | | NP_004010.1, |
| | | NP_004011.2, |
| | | NP_004012.1, |
| | | NP_004013.1, |
| | | NP_004014.1 |
| Ceroid | Late infantile | NP_000382.3 |
| lipofuscinosis | neuronal | |
| neuronal | ceroidlipo- | |
| 2 (CLN2) | fuscinosis or | |
| | Batten's | |
| | disease | |
| Neurturin | Parkinson's | NP_004549.1 |
| (NRTN) | disease | |
| N-acetylglucos- | Sanfilippo | NP_000254.2 |
| aminidase, alpha | syndrome | |
| (NAGLU) | (MPSIIIB) | |
| Iduronidase, | MPSI-Hurler | NP_000194.2 |
| alpha-l (IDUA) | | |
| Iduronate | MPSII- | NP_000193.1, |
| 2-sulfatase | Hunter | NP_001160022.1, |
| (IDS) | | NP_006114.1 |
| Glucuronidase, | MPSVII-Sly | NP_000172.2, |
| beta (GUSB) | | NP_001271219.1 |
| Hexosaminidase A, | Tay-Sachs | NP_000511.2 |
| α polypeptide | | |
| (HEXA) | | |
| Retinal pigment | Leber | NP_000320.1 |
| epithelium-specific | congenital | |
| protein 65 kDa | amaurosis | |
| (RPE65) | | |
| Factor IX (FIX) | Hemophilia B | NP_000124.1 |
| Adenine | progressive | NP_001142.2 |
| nucleotide | external | |
| translocator | ophthalmoplegia | |
| (ANT-1) | | |
| ApaLI | mitochondrial | YP_007161330.1 |
| | heteroplasmy, | |
| | myoclonic epilepsy | |
| | with ragged red | |
| | fibers (MERRF) or | |
| | mitochondrial | |
| | encephalomyopathy, | |
| | lactic acidosis, | |
| | and stroke-like | |
| | episodes (MELAS) | |
| NADH ubiquinone | Leber hereditary | YP_003024035.1 |
| oxidoreductase | optic | |
| subunit 4 (ND4) | | |
| very long- | very long-chain | NP_000009.1, |
| acyl-CoA | acyl-CoA | NP_001029031.1, |
| dehydrogenase | dehydrogenase | NP_001257376.1, |
| (VLCAD) | (VLCAD) | NP_001257377.1 |
| | deficiency | |
| short-chain | short-chain | NP_000008.1 |
| acyl-CoA | acyl-CoA | |
| dehydrogenase | dehydrogenase | |
| (SCAD) | (SCAD) | |
| | deficiency | |
| medium-chain | medium-chain | NP_000007.1, |
| acyl-CoA | acyl-CoA | NP_001120800.1, |
| dehydrogenase | dehydrogenase | NP_001272971.1, |
| (MCAD) | (MCAD) | NP_001272972.1, |
| | deficiency | NP_001272973.1 |
| Myotubularin 1 | X-linked | NP_000243.1 |
| (MTM1) | myotubular | |
| | myopathy | |
| Myophosphorylase | McArdle disease | NP_001158188.1, |
| (PYGM) | (glycogen | NP_005600.1 |
| | storage disease | |
| | type V, | |
| | myophosphorylase | |
| | deficiency) | |
| Lipoprotein lipase | LPL deficiency | NP_000228.1 |
| (LPL) | | |
| sFLT01 (VEGF/PlGF | Age-related | SEQ ID NO: 2, 8, |
| (placental growth | macular | 21, 23, or 25 of |
| factor) binding | degeneration | WO2009105669 |
| domain of human | | |
| VEGFR1/Flt-1 | | |
| (hVEGFR1) fused to | | |
| the Fc portion of | | |
| human IgG(1) through | | |
| a polyglycine linker) | | |
| Glucocerebrosidase | Gaucher | NP_000148.2, |
| (GC) | disease | NP_001005741.1, |
| | | NP_001005742.1, |
| | | NP_001165282.1, |
| | | NP_001165283.1 |
| UDP glucuronosyl- | Crigler-Najjar | NP_000454.1 |
| transferase 1 | syndrome | |
| family, polypep- | | |
| tide A1 (UGT1A1) | | |
| Glucose 6-phosphatase | GSD-Ia | NP_000142.2, |
| (G6Pase) | | NP_001257326.1 |
| Ornithine carbamoyl- | OTC | NP_000522.3 |
| transferase (OTC) | deficiency | |
| Cystathionine- | Homocystinuria | NP_000062.1, |
| beta-synthase | | NP_001171479.1, |
| (CBS) | | NP_001171480.1 |
| Factor VIII | Haemophilia | NP_000123.1, |
| (F8) | A | NP_063916.1 |
| Hemochromatosis | Hemochromatosis | NP_000401.1, |
| (HFE) | | NP_620572.1, |
| | | NP_620573.1, |
| | | NP_620575.1, |
| | | NP_620576.1, |
| | | NP_620577.1, |
| | | NP_620578.1, |
| | | NP_620579.1, |
| | | NP_620580.1 |
| Low density | Phenylketonuria | NP_000518.1, |
| lipoprotein | (PKU) | NP_001182727.1, |
| receptor | | NP_001182728.1, |
| (LDLR) | | NP_001182729.1, |
| | | NP_001182732.1 |
| Galactosidase, | Fabry disease | NP_000160.1 |
| alpha (AGA) | | |
| Phenylalanine | Hypercholes- | NP_000268.1 |
| hydroxylase | terolaemia or | |
| (PAH) | Phenylketonuria | |
| | (PKU) | |
| Propionyl CoA | Propionic | NP_000273.2, |
| carboxylase, | acidaemias | NP_001121164.1, |
| alpha polypeptide | | NP_001171475.1 |
| (PCCA) | | |

Other exemplary polypeptides or proteins of interest include adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphoring, semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is not AAV8. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). In some embodiments, the rAAV particle is a pseudotyped rAAV particle, which comprises (a) a nucleic acid vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some embodiments, the rAAV particle comprises a capsid that includes modified capsid proteins (e.g., capsid proteins comprising a modified VP3 region). Methods of producing modified capsid proteins are known in the art (see, e.g., US Patent Publication Number US20130310443, which is incorporated herein by reference in its entirety). In some embodiments, the rAAV particle comprises a modified capsid protein comprising a non-tyrosine residue (e.g., a phenylalanine) at a position that corresponds to a surface-exposed tyrosine residue in a wild-type capsid protein, a non-threonine residue (e.g., a valine) at a position that corresponds to a surface-exposed threonine residue in the wild-type capsid protein, a non-lysine residue (e.g., a glutamic acid) at a position that corresponds to a surface-exposed lysine residue in the wild-type capsid protein, a non-serine residue (e.g., valine) at a position that corresponds to a surface-exposed serine residue in the wild-type capsid protein, or a combination thereof. Exemplary surface-exposed lysine residues include positions that correspond to K258, K321, K459, K490, K507, K527, K572, K532, K544, K549, K556, K649, K655, K665, or K706 of the wild-type AAV2 capsid protein. Exemplary surface-exposed serine residues include positions that correspond to S261, S264, S267, S276, S384, S458, S468, S492, S498, S578, S658, S662, S668, S707, or S721 of the wild-type AAV2 capsid protein. Exemplary surface-exposed threonine residues include positions that correspond to T251, T329, T330, T454, T455, T503, T550, T592, T581, T597, T491, T671, T659, T660, T701, T713, or T716 of the wild-type AAV2 capsid protein. Exemplary surface-exposed tyrosine residues include positions that correspond to Y252, Y272, Y444, Y500, Y700, Y704, or Y730 of the wild-type AAV2 capsid protein.

Exemplary, non-limiting wild-type capsid protein sequences are provided below.

| Exemplary AAV1 capsid protein | |
|---|---|
| (SEQ ID NO: 14) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEEVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD EDKFFPMSGV MIFGKESAGA |
| 551 | SNTALDNVMI TDEEEIKATN PVATERFGTV AVNFQSSSTD PATGDVHAMG |
| 601 | ALPGMVWQDR DVYLQGPIWA KIPHTDGHFH PSPLMGGFGL KNPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |
| Exemplary AAV2 capsid protein | |
| (SEQ ID NO: 15) | |
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTTTQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDTNGVY SEPRPIGTRY LTRNL |
| Exemplary AAV3 capsid protein | |
| (SEQ ID NO: 16) | |
| 1 | MAADGYLPDW LEDNLSEGIR EWWALKPGVP QPKANQQHQD NRRGLVLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPF HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTTN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDTNGV YSEPRPIGTR YLTRNL |
| Exemplary AAV4 capsid protein | |
| (SEQ ID NO: 17) | |
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | QRLQGDTSFG GNLGRAVFQA KKRVLEPLGL VEQAGETAPG KKRPLIESPQ |
| 151 | QPDSSTGIGK KGKQPAKKKL VFEDETGAGD GPPEGSTSGA MSDDSEMRAA |
| 201 | AGGAAVEGGQ GADGVGNASG DWHCDSTWSE GHVTTTSTRT WVLPTYNNHL |
| 251 | YKRLGESLQS NTYNGFSTPW GYFDFNRFHC HFSPRDWQRL INNNWGMRPK |
| 301 | AMRVKIFNIQ VKEVTTSNGE TTVANNLTST VQIFADSSYE LPYVMDAGQE |
| 351 | GSLPPFPNDV FMVPQYGYCG LVTGNTSQQQ TDRNAFYCLE YFPSQMLRTG |
| 401 | NNFEITYSFE KVPFHSMYAH SQSLDRLMNP LIDQYLWGLQ STTTGTTLNA |
| 451 | GTATTNFTKL RPTNFSNFKK NWLPGPSIKQ QGFSKTANQN YKIPATGSDS |
| 501 | LIKYETHSTL DGRWSALTPG PPMATAGPAD SKFSNSQLIF AGPKQNGNTA |
| 551 | TVPGTLIFTS EEELAATNAT DTDMWGNLPG GDQSNSNLPT VDRLTALGAV |
| 601 | PGMVWQNRDI YYQGPIWAKI PHTDGHFHPS PLIGGFGLKH PPPQIFIKNT |
| 651 | PVPANPATTF SSTPVNSFIT QYSTGQVSVQ IDWEIQKERS KRWNPEVQFT |
| 701 | SNYGQQNSLL WAPDAAGKYT EPRAIGTRYL THHL |
| Exemplary AAV5 capsid protein | |
| (SEQ ID NO: 18) | |
| 1 | MSFVDHPPDW LEEVGEGLRE FLGLEAGPPK PKPNQQHQDQ ARGLVLPGYN |
| 51 | YLGPGNGLDR GEPVNRADEV AREHDISYNE QLEAGDNPYL KYNHADAEFQ |
| 101 | EKLADDTSFG GNLGKAVFQA KKRVLEPFGL VEEGAKTAPT GKRIDDHFPK |
| 151 | RKKARTEEDS KPSTSSDAEA GPSGSQQLQI PAQPASSLGA DTMSAGGGGP |
| 201 | LGDNNQGADG VGNASGDWHC DSTWMGDRVV TKSTRTWVLP SYNNHQYREI |
| 251 | KSGSVDGSNA NAYFGYSTPW GYFDFNRFHS HWSPRDWQRL INNYWGFRPR |
| 301 | SLRVKIFNIQ VKEVTVQDST TTIANNLTST VQVFTDDDYQ LPYVVGNGTE |
| 351 | GCLPAFPPQV FTLPQYGYAT LNRDNTENPT ERSSFFCLEY FPSKMLRTGN |
| 401 | NFEFTYNFEE VPFHSSFAPS QNLFKLANPL VDQYLYRFVS TNNTGGVQFN |
| 451 | KNLAGRYANT YKNWFPGPMG RTQGWNLGSG VNRASVSAFA TTNRMELEGA |
| 501 | SYQVPPQPNG MTNNLQGSNT YALENTMIFN SQPANPGTTA TYLEGNMLIT |
| 551 | SESETQPVNR VAYNVGGQMA TNNQSSTTAP ATGTYNLQEI VPGSVWMERD |
| 601 | VYLQGPIWAK IPETGAHFHP SPAMGGFGLK HPPPMMLIKN TPVPGNITSF |
| 651 | SDVPVSSFIT QYSTGQVTVE MEWELKKENS KRWNPEIQYT NNYNDPQFVD |
| 701 | FAPDSTGEYR TTRPIGTRYL TRPL |
| Exemplary AAV6 capsid protein | |
| (SEQ ID NO: 19) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |
| Exemplary AAV7 capsid protein | |
| (SEQ ID NO: 20) | |
| 1 | MAADGYLPDW LEDNLSEGIR EWWDLKPGAP KPKANQQKQD NGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP AKKRPVEPSP |
| 151 | QRSPDSSTGI GKKGQQPARK RLNFGQTGDS ESVPDPQPLG EPPAAPSSVG |
| 201 | SGTVAAGGGA PMADNNEGAD GVGNASGNWH CDSTWLGDRV ITTSTRTWAL |
| 251 | PTYNNHLYKQ ISSETAGSTN DNTYFGYSTP WGYFDFNRFH CHFSPRDWQR |
| 301 | LINNNWGFRP KKLRFKLFNI QVKEVTTNDG VTTIANNLTS TIQVFSDSEY |
| 351 | QLPYVLGSAH QGCLPPFPAD VFMIPQYGYL TLNNGSQSVG RSSFYCLEYF |
| 401 | PSQMLRTGNN FEFSYSFEDV PFHSSYAHSQ SLDRLMNPLI DQYLYYLART |
| 451 | QSNPGGTAGN RELQFYQGGP STMAEQAKNW LPGPCFRQQR VSKTLDQNNN |
| 501 | SNFAWTGATK YHLNGRNSLV NPGVAMATHK DDEDRFFPSS GVLIFGKTGA |
| 551 | TNKTTLENVL MTNEEEIRPT NPVATEEYGI VSSNLQAANT AAQTQVVNNQ |
| 601 | GALPGMVWQN RDVYLQGPIW AKIPHTDGNF HPSPLMGGFG LKHPPPQILI |
| 651 | KNTPVPANPP EVFTPAKFAS FITQYSTGQV SVEIEWELQK ENSKRWNPEI |
| 701 | QYTSNFEKQT GVDFAVDSQG VYSEPRPIGT RYLTRNL |
| Exemplary AAV8 capsid protein | |
| (SEQ ID NO: 21) | |
| 1 | MAADGYLPDW LEDNLSEGIR EWWALKPGAP KPKANQQKQD DGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPARK RLNFGQTGDS ESVPDPQPLG EPPAAPSGVG |
| 201 | PNTMAAGGGA PMADNNEGAD GVGSSSGNWH CDSTWLGDRV ITTSTRTWAL |
| 251 | PTYNNHLYKQ ISNGTSGGAT NDNTYFGYST PWGYFDFNRF HCHFSPRDWQ |
| 301 | RLINNNWGFR PKRLSFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFQFTYTFED VPFHSSYAHS QSLDRLMNPL IDQYLYYLSR |
| 451 | TQTTGGTANT QTLGFSQGGP NTMANQAKNW LPGPCYRQQR VSTTTGQNNN |
| 501 | SNFAWTAGTK YHLNGRNSLA NPGIAMATHK DDEERFFPSN GILIFGKQNA |
| 551 | ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL |
| 651 | IKNTPVPADP PTTFNQSKLN SFITQYSTGQ VSVEIEWELQ KENSKRWNPE |
| 701 | IQYTSNYYKS TSVDFAVNTE GVYSEPRPIG TRYLTRNL |
| Exemplary AAV9 capsid protein | |
| (SEQ ID NO: 22) | |
| 1 | MAADGYLPDW LEDNLSEGIR EWWALKPGAP QPKANQQHQD NARGLVLPGY |
| 51 | KYLGPGNGLD KGEPVNAADA AALEHDKAYD QQLKAGDNPY LKYNHADAEF |
| 101 | QERLKEDTSF GGNLGRAVFQ AKKRLLEPLG LVEEAAKTAP GKKRPVEQSP |
| 151 | QEPDSSAGIG KSGAQPAKKR LNFGQTGDTE SVPDPQPIGE PPAAPSGVGS |
| 201 | LTMASGGGAP VADNNEGADG VGSSSGNWHC DSQWLGDRVI TTSTRTWALP |
| 251 | TYNNHLYKQI SNSTSGGSSN DNAYFGYSTP WGYFDFNRFH CHFSPRDWQR |
| 301 | LINNNWGFRP KRLNFKLFNI QVKEVTDNNG VKTIANNLTS TVQVFTDSDY |
| 351 | QLPYVLGSAH EGCLPPFPAD VFMIPQYGYL TLNDGSQAVG RSSFYCLEYF |
| 401 | PSQMLRTGNN FQFSYEFENV PFHSSYAHSQ SLDRLMNPLI DQYLYYLSKT |
| 451 | INGSGQNQQT LKFSVAGPSN MAVQGRNYIP GPSYRQQRVS TTVTQNNNSE |
| 501 | FAWPGASSWA LNGRNSLMNP GPAMASHKEG EDRFFPLSGS LIFGKQGTGR |
| 551 | DNVDADKVMI TNEEEIKTTN PVATESYGQV ATNHQSAQAQ AQTGWVQNQG |
| 601 | ILPGMVWQDR DVYLQGPIWA KIPHTDGNFH PSPLMGGFGM KHPPPQILIK |
| 651 | NTPVPADPPT AFNKDKLNSF ITQYSTGQVS VEIEWELQKE NSKRWNPEIQ |
| 701 | YTSNYYKSNN VEFAVNTEGV YSEPRPIGTR YLTRNL |
| Exemplary AAV10 capsid protein | |
| (SEQ ID NO: 23) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERFFPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL |

In some embodiments, the modified capsid protein comprises a non-tyrosine (e.g., a phenylalanine) residue at one or more of or each of Y705 and Y731 of a wild-type AAV3 capsid protein. In some embodiments, the modified capsid protein comprises a non-serine residue (e.g., valine) and/or a non-threonine residue (e.g., valine) at one or more of or each of S663 and T492 of a wild-type AAV3 capsid protein. In some embodiments, the modified capsid protein comprises a non-serine residue (e.g., valine), a non-threonine residue (e.g., valine), and/or a non-lysine residue (e.g., arginine) at one or more of or each of S663, T492V and K533 of a wild-type AAV3 capsid protein. In some embodiments, the modified capsid protein comprises a non-tyrosine (e.g., a phenylalanine) residue, non-serine residue (e.g., valine), a non-threonine residue (e.g., valine), and/or a non-lysine residue (e.g., arginine) at one or more of or each of Y705, Y731, S663, T492V and K533 of a wild-type AAV3 capsid protein.

In some embodiments, the modified capsid protein comprises a non-tyrosine residue and/or a non-threonine residue at one or more of or each of Y705, Y731, and T492 of a wild-type AAV6 capsid protein (see sequence below with Y705, Y731, and T492 positions underlined, bolded and italicized). In some embodiments, the non-tyrosine residue is phenylalanine and the non-threonine residue is valine.

| (SEQ ID NO: 24) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYFLNRTQ |
| 451 | NQSGSAQNKD LLFSRGSPAG MSVQPKNWLP GPCYRQQRVS K [symbol] KTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSN [symbol] AKSAN VDFTVDNNGL YTEPRPIGTR [symbol] LTRPL |

In some embodiments, two rAAV particles are contemplated. In some embodiments, the first rAAV particle comprises a nucleic acid vector as described herein (e.g., comprising a one or more heterologous nucleic acid regions comprising a sequence encoding a protein or polypeptide of interest flanked by ITR sequences), and the second rAAV particle comprises a second nucleic acid vector that contains a region that encodes a Rep protein as described herein. In some embodiments, the second nucleic acid vector further contains ITR sequences flanking the region encoding the Rep protein.

Other aspects of the disclosure relate to a nucleic acid vector, such as a recombinant nucleic acid vector comprising a nucleic acid region encoding a Rep protein as described herein. In some embodiments, the nucleic acid vector further comprises the one or more heterologous nucleic acid regions comprising a sequence encoding a protein or polypeptide of interest wherein the one or more heterologous nucleic acid regions are flanked by ITR sequences. In some embodiments, the nucleic acid region encoding the Rep protein is also flanked by the ITR sequences. In some embodiments, the nucleic acid region encoding the Rep protein is outside of the region flanked by the ITR sequences. In some embodiments, the nucleic acid vector is provided in a form suitable for inclusion in a rAAV particle, such as a single-stranded or self-complementary nucleic acid. In some embodiments, the nucleic acid vector is provided in a form suitable for use in a method of producing rAAV particles. For example, in some embodiments, the nucleic acid vector is a plasmid (e.g., comprising an origin of replication (such as an E. coli ORI) and optionally a selectable marker (such as an Ampicillin or Kanamycin selectable marker)).

Methods of producing rAAV particles and nucleic acid vectors are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid containing the nucleic acid vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the packaging is performed in a helper cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprises rep and/or cap genes that encode the Rep protein and/or Cap proteins for use in a method described herein. In some embodiments, the packaging is performed in vitro.

Other aspects relate to a helper cell expressing a Rep protein of a first serotype (e.g., AAV3, 5 or 6) and AAV capsid proteins of the same serotype. In some embodiments, the helper cell is a mammalian or insect cell as described herein. In some embodiments, the helper cell further comprises a nucleic acid vector as described herein, e.g., comprising ITRs of the first serotype. It is to be understood that any configuration for delivering a combination of nucleic acid vector, Rep protein, and AAV capsid proteins to a helper or packaging cell can be used as aspects of the disclosure are not limited in this respect.

In some embodiments of a method provided herein, a plasmid containing the nucleic acid vector is combined with one or more helper plasmids, e.g., that contain a rep gene of a first serotype and a cap gene of the same serotype or a different serotype, and transfected into a helper cell line such that the rAAV particle is packaged.

In some embodiments, the one or more helper plasmids include a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV3, AAV5, or AAV6 and the cap gene is derived from AAV2, AAV3, AAV5, or AAV6 and may include modifications to the gene in order to produce the modified capsid protein described herein. Exemplary AAV Rep protein sequences are provided herein. In some embodiments, the rep gene is a rep gene derived from AAV2 or AAV6 and the cap gene is derived from AAV6 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

The disclosure also contemplates host cells that comprise at least one of the disclosed rAAV particles or nucleic acid vectors described herein and optionally further comprise a Rep protein (e.g., in the form of a second rAAV particle, an mRNA, or the protein itself). Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

The disclosure also provides compositions comprising one or more of the disclosed rAAV particles or preparations. In some embodiments, the rAAV preparation comprises an rAAV particle comprising a nucleic acid vector containing ITRs of a first serotype (e.g., AAV3, AAV5, or AAV6) and capsid proteins encapsidating the nucleic acid vector. In some embodiments, the capsid proteins are of the first serotype (e.g., AAV3, AAV5, or AAV6). In some embodiments, the preparation has at least a four-fold higher transduction efficiency (e.g., in a human hepatocellular carcinoma cell line, such as Huh7) compared to a preparation prepared using a nucleic acid vector containing AAV2 ITRs.

The disclosure also contemplates host cells that comprise at least one of the disclosed rAAV particles or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cancer cell. In some embodiments, the host cell is a liver cell, such as a liver cancer cell.

### Subjects

Aspects of the disclosure relate to methods and preparations for use with a subject, such as human or non-human primate subjects, a host cell in situ in a subject, or a host cell derived from a subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of complete blood count, hemoglobin electrophoresis, Fe Binding Capacity, urine urobilin and urobilogen, peripheral blood smear, hematocrit, and genetic testing.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art.

In some embodiments, the subject has or is suspected of having a disease that may be treated with gene therapy. In some embodiments, the subject has or is suspected of having a disease provided in Table 1.

In some embodiments, the subject has or is suspected of having a disease that may be treated with gene therapy. In some embodiments, the subject has or is suspected of having a proliferative disease, such as cancer. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is liver cancer. Exemplary liver cancers include, but are not limited to, hepatocellular carcinoma (HCC), cholangiocarcinoma, angiosarcoma, and hepatoblastoma. Subject having cancer can be identified by the skilled medical practitioner, e.g., using methods known in the art including biopsy, cytology, histology, endoscopy, X-ray, Magnetic Resonance Imaging (MRI), ultrasound, CAT scan (computerized axial tomography), genetic testing, and tests for detection of tumor antigens in the blood or urine.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Site-Specific Integrating Recombinant AAV Vectors for Human Hematopoietic Stem Cell Transduction

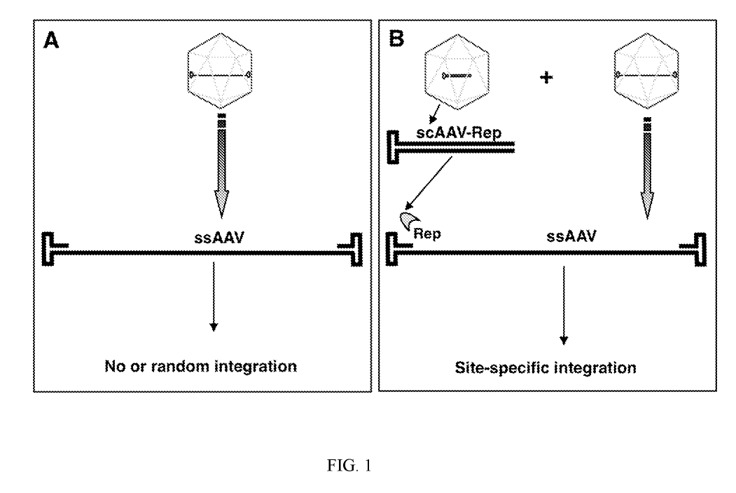

Previously, studies have described the remarkable site-specificity of integration of the wild-type (wt) adeno-associated virus 2 (AAV2) genome into the long-arm of chromosome 19 in human cells (Proc. Natl. Acad. Sci., USA, 87: 2211-2215, 1990; EMBO J., 10: 3941-3950, 1991). However, this specificity of integration is lost with recombinant AAV vectors (Hum. Gene Ther., 8: 275-284, 1997). Interestingly, the site-specificity of integration can be restored to recombinant AAV genomes, if the AAV Rep proteins are supplied in trans (J. Virol., 83: 11655-11664, 2009). Of the 10 most commonly used AAV serotype vectors, the AAV6 vectors transduce primary human hematopoietic stem cells (HSCs) most efficiently (Cytotherapy, 15: 986-996, 2013; PLoS One, 8: e58757, 2013). The use of recombinant lentiviral vectors in a clinical trial has led to activation of a cellular proto-oncogene, frequently associated with pre-leukemia, in a patient due to random integration of the lentiviral genome (Nature, 467: 318-322, 2010). It is hypothesized that AAV Rep-mediated site-specific integration of a therapeutic gene delivered to human HSCs using optimized AAV6 serotype vectors will eliminate or minimize the possibility of insertional mutagenesis. Thus, primary human HSCs are transduced with a therapeutic AAV6 vector and AAV Rep proteins are delivered, either using a dual vector approach, or following mRNA transfection, to achieve site-specific integration of the therapeutic gene in human HSCs (FIG. 1).

Studies are underway to assess site-specific integration of a normal human β-globin gene in primary human HSCs, which would prove to be a safer alternative for the potential gene therapy of a wide variety of human diseases involving the hematopoietic system.

### Example 2. Novel Recombinant AAV3 Serotype Vectors for Gene Therapy of Human Liver Cancers

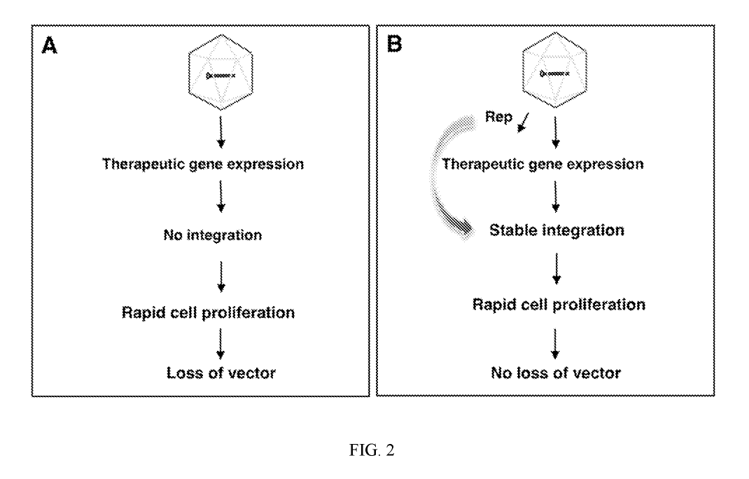

Although recombinant AAV3 serotype vectors were largely ignored previously, owing to their poor transduction efficiency in all cells and tissues examined, initial observations of their selective tropism for human liver cancer cell lines and primary human hepatocytes (Mol Genet Metabol., 98: 289-299, 2009; Hum Gene Ther., 21: 1741-1747, 2010; Gene Ther., 19: 375-84, 2012), has led to renewed interest in this serotype, since AAV3 vectors and their variants have recently proven to be extremely efficient in targeting human and non-human primate hepatocytes in vitro as well as in vivo (Mol Ther., 22: S2, 2014; Mol Ther., 22: S91, 2014; Nature, 506: 382-386, 2014). More recently, AAV3 vectors were developed expressing a therapeutic gene to target human liver tumors in a murine xenograft model in vivo (Hum Gene Ther., 25: 1023-1034, 2014). However, the therapeutic benefit was lost due to rapid tumor growth and dilution of the vector genomes since these genomes failed to integrate into chromosomal DNA (FIG. 2A). Interestingly, AAV2 Rep proteins possess not only cytostatic/cytotoxic properties, but they also mediate site-specific integration of the recombinant AAV genome into human cells (FIG. 2B). It is also likely that the Rep proteins from three additional AAV serotypes, AAV3, AA5, and AAV6, that are currently available, also possess these properties. Thus, the use of AAV Rep genes to target human liver cancers is likely to provide the added benefits that unlike their cellular counterparts, their expression will be less amenable to the control mechanisms in the tumor, as well as stable integration of the vector genomes, thereby circumventing the problem of vector dilution in rapidly growing tumors. It is proposed to generate capsid-optimized AAV3 vectors expressing the Rep proteins from AAV2, AAV3, AAV5, and AAV6 to determine the most efficient serotype for targeting human liver tumors in murine xenograft models in vivo. Since Rep-induced apoptosis appears to be p53-independent, Rep is expected to function in human liver cancers of multiple etiologies with varying p53 status. The vectors generated for this purpose may also allow for the study of gene targeting and apoptosis therapy of other cancers as well.

Thus, the availability of these novel AAV3-Rep serotypes vectors, will have significant implications in their optimal use in human gene therapy of cancer in general, liver tumors in particular.

### Example 3: Strategies to Achieve High-Titer, High Potency Recombinant AAV Serotype Vectors

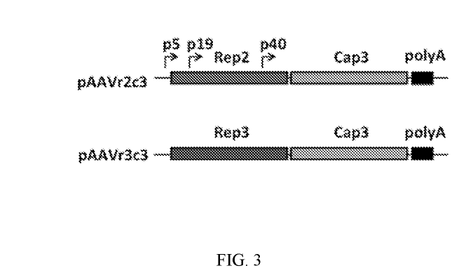

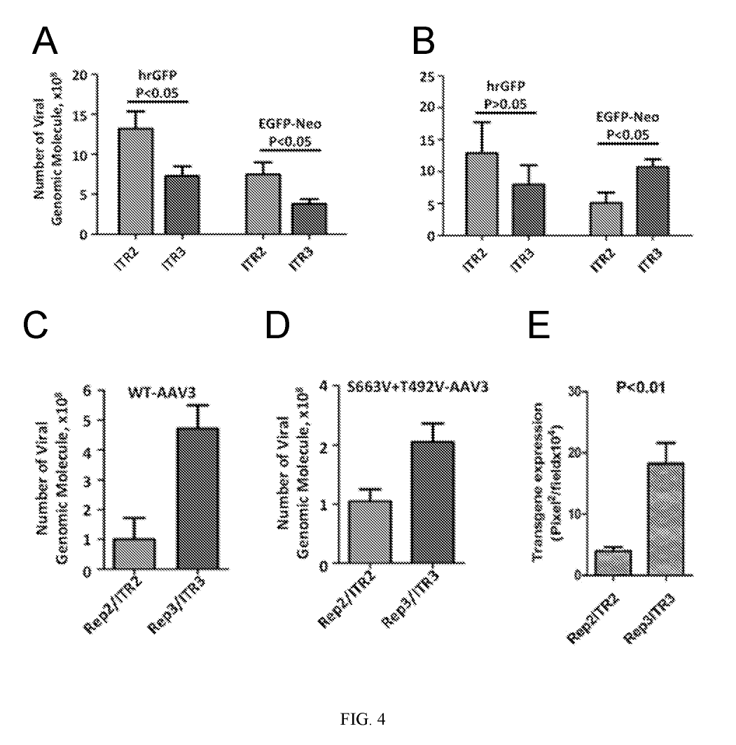

Recombinant AAV serotype vectors are generated using expression cassettes flanked by the AAV2 inverted terminal repeats (ITRs) and the AAV2 Rep proteins. Previously, it was reported that the combination of ITR8 with AAV8 capsids (AAV8/8) resulted in vectors that led to at least 2-fold increase in transgene expression in mouse liver, compared with AAV8 capsids pseudotyped with ITR2 (AAV2/8) vectors (Mol. Ther. 11: S156, 2005). However, another group reported that the ITRs from AAV serotypes 1-6 were interchangeable when they are packaged into AAV8 capsids (AAV1/8-AAV6/8), and played no role in transgene expression in murine hepatocytes in vivo (J. Virol., 80: 426-439, 2006). The study herein evaluated the relative contribution of the cis-acting ITR from AAV3 (ITR3), as well as the trans-acting Rep proteins from AAV3 (Rep3) in the recombinant AAV3 vector production and transduction. To this end, two helper plasmid were utilized: pAAVr2c3, which carried rep2 and cap3 genes and pAAVr3c3, which carried rep3 and cap3 genes (FIG. 3). Plasmid transfection assays revealed that both AAV2 and AAV3 Rep and Cap proteins were expressed at similar levels in the presence of adenoviral helper genes. Two sets of single-stranded AAV vector constructs were also generated carrying an expression cassette containing the EGFP reporter gene flanked by either ITR2 or ITR3. Plasmid transfections of the ITR2- or the ITR3-containing expression cassettes into cultured cells also revealed no difference in the extent of transgene expression. In contrast, viral genome rescue and replication assays indicated more efficient replication of the viral genomes flanked by ITR2 in the presence of Rep2 (FIG. 4A) when compared to viral genomes flanked by ITR2 in the presence of Rep3 (FIG. 4B). Similarly, the extent of replication of the viral genome flanked by ITR3 was higher in the presence of Rep3 (FIG. 4B) when compared to the viral genome flanked by ITR3 in the presence of Rep2 (FIG. 4A). Low-molecular-mass DNA was isolated 72 hours post-transfection, followed by DpnI digestion for 4 hours. The DNA samples were then subjected to qPCR assays for the quantification of viral genome copy number. It is evident that in the presence of Rep2, both ITR2-containing plasmids resulted in a higher level of viral genome copy numbers. A higher viral genome copy number of ITR2/3-hrGFP was also observed compared with ITR2/3-EGFP-Neo, regardless of the origin of ITRs, which was most likely due to the sub-genomic length of the viral DNA. Interestingly, in the presence of Rep3, whereas the same origin of ITR had no significant effect on genome replication of shorter viral DNA, it clearly produced a higher number of viral genome copy number when the genome size was similar to the WT AAV DNA. Thus, in all subsequent experiments, the set of longer viral genomes, pITR2/3-EGFP-Neo, was used to examine the potential benefit of Rep3 and ITR3.

From the triple-transfected HEK293 cells, crude lysates were prepared 72 hours post-transfection. Samples were treated with both Benzonase and Universal Nuclease for 4 hours, followed by extraction of viral encapsidated genomic DNA. The negative controls of this assay included HEK293 cells that were trip-transfected with pITR2/3-EGFP-Neo, pHelper and a plasmid containing the AAV2 rep gene but no cap genes. In the negative control cells, viral genome replication occurs, but no viral capsid proteins are expressed. The results of viral encapsidation assays revealed that the encapsidated viral genomes in the negative control group were below the detection limitation by qPCR assays (data not shown). Importantly, the use of Rep3 and ITR3 to produce AAV3 vectors yielded ˜4-fold or ˜5 fold higher titers, compared with the group in which Rep2 and ITR2 were used (FIG. 4C). To validate these observations, viral encapsidation assays were performed using S663V+T492V-AAV3 capsids. The results also showed approximately 2-fold increase in vector titers when Rep3 and ITR3 were used (FIG. 4D). Next, purified viral stocks of recombinant AAV3 vectors were generated and designated as Rep2ITR2 and Rep3ITR3, respectively. Interestingly, the transduction efficiency of Rep3-ITR3 AAV3 vectors was ˜4-fold higher than that of Rep2-ITR2 AAV3 vectors in a human hepatocellular carcinoma cell line, Huh7, under identical conditions (FIG. 4E). In addition, Western blot assays and Southern blot assays to determine viral capsid proteins and DNA genomes, respectively, were performed to corroborate the transduction results. Additional data investigating transduction efficiencies was obtained.

### Example 4: AAV3 Vectors Produced by Homologous Rep Proteins and ITRs Lead to Higher Transduction Efficiency

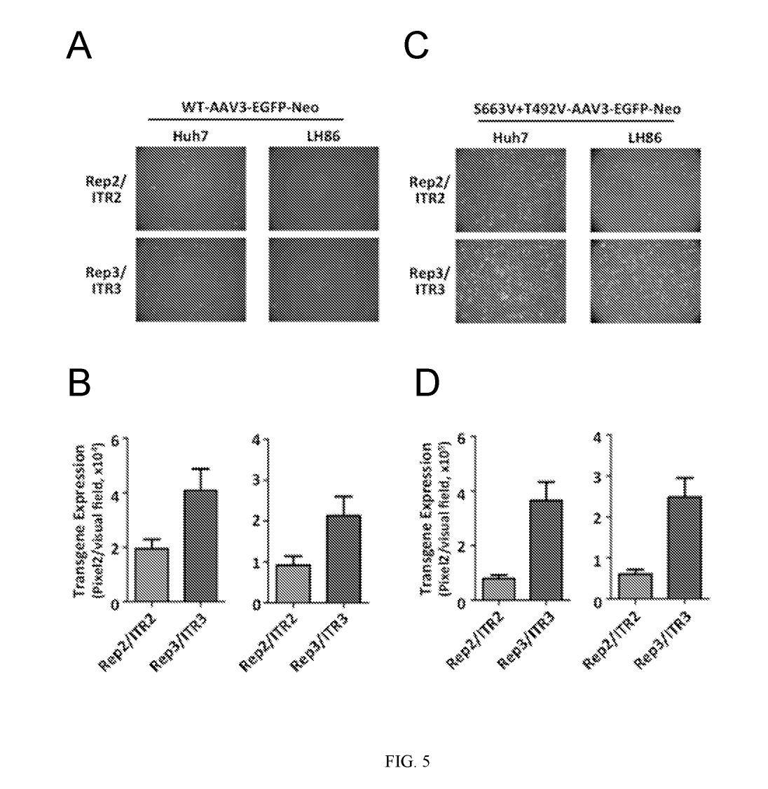

Purified WT-AAV3 and S663V+T492V-AAV3 vectors containing the EGFP-Neo transgene cassette were produced either in the presence of Rep2/ITR2 or Rep3/ITR3. Two human hepatocellular carcinoma (HCC) cell lines, Huh7 and LH86, were transduced with these vectors under identical conditions, and transgene expression was determined 72 hours post-transduction. The S663V+T492V-AAV3 vectors led to >10-fold increase in the transduction efficiency. Interestingly, the data indicated that the WT-AAV3 vectors, which were generated with ITR3, Rep3, and Cap3, transduced both human HCC cell lines ˜2-fold more efficiently than those generated with ITR2, Rep2, and Cap3 (FIGS. 5A and 5B). Similar results were also obtained when S663V+T492V-AAV3 vectors were generated with Rep2/ITR2 and Rep3/ITR3, and were each used to infect the human HCC cell lines (FIGS. 5C and 5D).

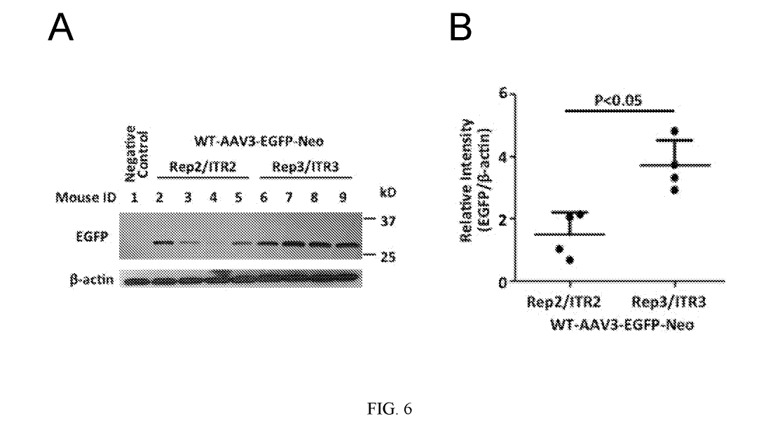

To further corroborate the results of in vitro experiments, Huh7 tumor-bearing immune-deficient mouse models were generated. When the tumor grew to 0.5 cm in diameter, AAV3-EGFP-Neo vectors, produced either with Rep2/ITR2 (FIG. 6A, lanes 2 to 5) or with Rep3/ITR3 (FIG. 6A, lanes 6 to 9), were injected intra-tumorally with 1×10¹¹ vgs/tumor. Forty-eight hours post-vector administration, tumors were obtained, and transgene expression was evaluated by Western blot assays. A tumor without vector injection (FIG. 6A, lane 1) was used as a negative control. These results, consistent with those of the in vitro experiments, indicated that the AAV3 vectors generated with Rep3/ITR3 transduced human liver tumors in vivo ˜2-fold more efficiently than their counterpart produced with Rep2/ITR2 (FIG. 6B). Taken together, the experimental data suggest the combined use of Rep3 and ITR3 leads to generation of recombinant AAV3 vectors at higher titers as well as higher transduction efficiency in human liver cancer cell lines in vitro and in human liver tumors in a murine xenograft model in vivo.

In summary, these studies document that the combined use of the AAV3 ITRs, AAV3 Rep proteins, and AAV3 capsids led to the production of recombinant AAV3 vectors with higher titers and with higher transduction efficiency, and suggests that the use of homologous ITRs, Rep proteins, and capsids are similarly likely to be applicable to other AAV serotypes vectors, such as AAV5 and AAV6, for their optimal use in human gene therapy.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of promoting site-specific nucleic acid integration into a host genome, the method comprising: delivering a recombinant adeno-associated virus (rAAV) particle comprising a nucleic acid vector to a host cell in the presence of a Rep protein.

2. The method of claim 1, wherein the nucleic acid vector comprises AAV2 inverted terminal repeats (ITRs) or AAV6 ITRs.

3. The method of any prior claim, wherein rAAV particle is a AAV6 particle.

4. The method of any prior claim, wherein the host cell is a human cell.

5. The method of any prior claim, wherein the host cell is a stem cell.

6. The method of any prior claim, wherein the host cell is a liver, muscle, brain, eye, pancreas, kidney, or hematopoietic stem cell.

7. The method of any prior claim, wherein the host cell is ex vivo.

8. The method of any prior claim, wherein the host cell is in situ in a host.

9. The method of any prior claim, wherein the nucleic acid vector encodes the Rep protein.

10. The method of any prior claim, wherein the Rep protein is delivered to the host cell separately from the nucleic acid vector.

11. The method of any prior claim, wherein the Rep protein is expressed from a second nucleic acid that is delivered to the host cell.

12. The method of claim 11, wherein the second nucleic acid is an mRNA that is transiently transfected into the host cell.

13. The method of claim 11, wherein the second nucleic acid is transfected into the host cell in a viral particle.

14. The method of any prior claim, wherein the nucleic acid vector encodes a therapeutic protein.

15. The method of claim 14, wherein the therapeutic protein is human β-globin.

16. The method of any prior claim, wherein the Rep protein is an AAV2 or AAV6 Rep protein.

17. The method of claim 3, wherein the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination thereof.

18. The method of claim 17, wherein the modified capsid protein comprises a non-tyrosine residue and/or a non-threonine residue at one or more of or each of Y705, Y731, and T492 of a wild-type AAV6 capsid protein.

19. The method of claim 17 or 18, wherein the non-tyrosine residue is phenylalanine and the non-threonine residue is valine.

20. A method of treating a proliferative disease, the method comprising administering an rAAV particle comprising a nucleic acid vector that encodes a Rep protein to a subject having the proliferative disease.

21. The method of claim 20, wherein the rAAV particle is a recombinant AAV3, AAV5, or AAV6 particle.

22. The method of any one of claims 20-21, wherein the Rep protein is an AAV3, AAV5, or AAV6 Rep protein.

23. The method of claim 22, wherein the nucleic acid vector comprises AAV3 inverted terminal repeats (ITRs), AAV5 ITRs, or AAV6 ITRs.

24. The method of any one of claims 20-23, wherein the proliferative disease is cancer.

25. The method of claim 24, wherein the cancer is liver cancer.

26. The method of claim 25, wherein the nucleic acid vector comprises a human alpha-fetoprotein (AFP) promoter.

27. The method of any one of claims 24 to 26, wherein the nucleic acid vector further encodes a therapeutic protein or nucleic acid.

28. The method of claim 27, wherein the therapeutic protein or nucleic acid is selected from a caspase, Bcl2, BAX, p53, retinoblastoma (RB), thymidine kinase (TK), pyruvate dehydrogenase (PDH) E1α, β-catenin/Yes-associated protein 1 (YAP1)-siRNA, survivin siRNA, Parvovirus B19 non-structural protein 1 (NS1) and trichosanthin (TCS).

29. A method of producing an rAAV composition, the method comprising packaging a recombinant AAV nucleic acid vector comprising ITRs of a first serotype in the presence of a) a Rep protein of the same serotype and b) AAV capsid proteins, wherein the first serotype is not AAV2 or AAV8.

30. The method of claim 29, wherein the AAV capsid proteins are of the same serotype as the ITRs and Rep protein.

31. The method of any one of claims 29-30, wherein the first serotype is AAV3, AAV5 or AAV6.

32. The method of any one of claims 29-31, wherein the recombinant AAV nucleic acid vector encodes a therapeutic protein.

33. The method of any one of claims 29-32, wherein the therapeutic protein is selected from the group consisting of adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphorins, semaphorin receptors, serotonin transport proteins, serotonin uptake inhibitors, serotonin receptors, serpins, serpin receptors, and tumor suppressors.

34. The method of claim 29, wherein the packaging is performed in a helper cell.

35. The method of claim 29, wherein the packaging is performed in vitro.


SEQUENCE LISTING (24 sequences)

SEQ ID NO: 1 - DNA, 145 bp, Adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 146 bp, Adeno-associated virus 3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 3 - DNA, 167 bp, Adeno-associated virus 5
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 4 - DNA, 142 bp, Adeno-associated virus 6
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 5 - protein, 621 aa, Artificial Sequence
  Synthetic Polypeptide
TAGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 6 - protein, 624 aa, Artificial Sequence
  Synthetic Polypeptide
MPGFYEIVLKVPSDLDERLPGISNSFVNWVAEKEWDVPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFEFELTRRLDHDFGKVTKQEVKDFFRWASDHVTDVAHEFYVRKGGAKKRPASNDADVSEPKRECTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 7 - protein, 610 aa, Artificial Sequence
  Synthetic Polypeptide
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 8 - protein, 623 aa, Artificial Sequence
  Synthetic Polypeptide
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 9 - DNA, 275 bp, Artificial Sequence
  Synthetic Polynucleotide
agtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacacc

SEQ ID NO: 10 - DNA, 510 bp, Artificial Sequence
  Synthetic Polynucleotide
ggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaataacacc

SEQ ID NO: 11 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 12 - protein, 147 aa, Homo sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 13 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFAQLSELHCDKLHVDPENFRLLGNVLVCVLAHHFGKEFTPPVQAAYQKVVAGVANALAHKYH

SEQ ID NO: 14 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 735 aa, Adeno-associated virus 2 [sequence omitted]

SEQ ID NO: 16 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 17 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 18 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 19 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 20 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 21 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 24 - protein, 736 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYFLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL